(19) United States
(12) Patent Application Publication
(10) Pub. No.: US 2001/0018614 A1
(43) Pub. Date: Aug. 30, 2001

(54) Implants for orthopedic applications

(76) Inventor: John R. Bianchi, Alachua, FL (US)

Correspondence Address: Timothy H. Van Dyke, Bencen & Van Dyke, P.A., 1630 Hillcrest Street, Orlando, FL, 32803, US

(21) Appl. No.: 09/750,192

(22) Filed: Dec. 28, 2000

Related U.S. Application Data
(63) Continuation-in-part of application No. 09/268,812, filed on Mar. 16, 1999, now abandoned.
(63) Continuation-in-part of application No. 09/563,604, filed on May 2, 2000.

(51) Int. Cl. A61F 2/28; B29C 39/02; A61F 2/30; A61F 2/32

(52) U.S. Cl. 623/16.11; 264/460; 264/109; 264/118; 623/18.11; 623/22.11

(57) ABSTRACT

An implant and a method for making and using the implant are disclosed for the repair of bone defects or voids, including defects or voids in the acetabular cup. The implant shapes and compositions of this invention provide advantages not present in impaction grafts and like implants known in the art. Also disclosed is an osteogenic, cross-linked composite implant, and methods of producing the same.

### BACKGROUND OF THE INVENTION

1. Field of the Invention

This invention relates to an implant and methods for making and using the implant to fill void defects in bone and to accomplish orthopedic fusions.

2. Background Information

In the field of orthopedics, it is desirous to be able to fill bony defects and to be able to fuse joints together using grafting procedures. One procedure that is frequently required is the repair of skeletal void defects. In particular, it is frequently required that bony defects be filled or repaired after trauma or disease has destroyed the native bone. This need may arise from trauma, as in a compound or complex fracture, through removal of diseased tissue, as in, for example, removal of a cancerous growth, or any of a number of other degenerative or damaging conditions. It is common practice in spinal surgery to effect the fusion of adjacent vertebrae by placing bone graft between the vertebrae. This need may arise from a condition such as severe scoliosis, from trauma in which the back is severely damaged, or in the common instance of degenerative disk disease.

Prior to the present invention, the filling of bone defects was usually accomplished through the use of metallic fixation and reinforcement devices or the combination of metallic devices with autograft or allograft.

Recurrent problems in the methods known in the art are the lack of incorporation of the metallic graft materials, the pain associated with autograft harvest, the lack of sufficient amounts of autograft for harvesting, the labor-intensive nature of autograft and allograft preparation, and the relatively poor performance of commonly acquired allografts.

A recurring problem in the methods known in the art for repairing, for example, the acetabular surface is that frequently, upon insertion into the acetabulum of metallic or polymeric implant materials, voids remain between the back surface of the implant and the pelvic bone remaining in the original femoral socket.

In one method known in the art, generally referred to as “impaction grafting” (see, for example, Elting, et al., Clinical Orthopaedics and Related Research, 319:159-167, 1995), compressed morselized cancellous allograft bone is used to fashion implants for insertion, for example, into the intramedullary canal of recipients. However, problems associated with that technique include subsidence and the need to use synthetic “glues” such as polymethylmethacrylate. While cortical cancellous chips combined with metallic mesh and circlage wires have been used successfully to fill voids in the acetabulum and proximal femur, and while incorporation of bone chips and de novo bone formation at the impaction grafting site has been observed, cortical-cancellous chips handle poorly. The chips tend to behave like gravel and do not stay in the location into which they are placed unless enclosed by wire mesh or another retaining device. Furthermore, when methyl methacrylate or like cement is pressurized in impaction grafting, large amounts of bone chips become sequestered and therefore are biologically inactive.

In one recent patent, (see U.S. Pat. No. 5,824,078 and references cited therein), an apparatus was described for fashioning composite allograft by impaction of cancellous bone and added cement to form acetabular cups. These methods are limited in applicability in that the impacted implant, once formed, is no longer moldable and has limited pliability. The result of such inflexibility is that voids remain, even after the impacted graft is positioned in an appropriate location in a recipient. In addition, the impaction procedure itself requires specialized equipment (such as the rack-and-pinion device to which the 5,824,078 patent is directed) or time consuming in-surgery impaction of bone particles (see the Elting et al., article, which describes a six-step, in-situ, procedure which requires iterative packing and tamping of bone particles).

In U.S. Pat. No. 5,439,684, methods of making variously shaped pieces of demineralized swollen bone are disclosed. The shaped bone pieces are composed of large machined pieces of bone of specific shape and are thus not moldable and are not composed of cortical-cancellous bone chips.

This invention provides a solution to the above-noted, long-standing problems by providing specific shapes and compositions of biomaterials for filling of tissue voids, in particular in bony tissue, in an easy to use and effective format.

### BRIEF DESCRIPTION OF THE DRAWINGS

FIG. 1 is a representation of a first embodiment of the invention, wherein a disk-shaped bioimplant is provided for insertion into the acetabular socket or other location to fill voids that remain upon insertion of a metallic or other implant.

FIG. 2A is a representation of a second embodiment of the invention, wherein a substantially disk-shaped bioimplant is provided, but wherein a sector of the disk-shaped implant has either been removed or has not been included when initially created, so that upon insertion into the acetabluar socket, a substantially cone-shaped or hemisphere-shaped implant, FIG. 2B, is formed.

FIG. 3 provides representations of a number of further embodiments of the invention: FIG. 3A depicts a thin “U”-shaped implant useful in knee revision surgeries; FIG. 3B depicts a thicker “U”-shaped implant useful in spinal fusion procedures; FIG. 3C depicts a thin oval implant useful in knee revision and other surgical procedures; FIG. 3D depicts an implant shape useful in posterior lumbar interbody fusion (“PLIF”) procedures; FIG. 3E depicts a dowel shaped implant, useful in spinal and joint fusions; FIG. 3F depicts a tapered dowel shaped implant, useful in spinal and joint fusions.

FIG. 4 provides representations of a number of further embodiments of the invention: FIG. 4A depicts a femoral or tibial ring shaped implant useful in interbody fusion procedures; FIG. 4B depicts a round, plug-shaped implant useful in cranial burr-hole repairs; FIG. 4C depicts a thin “U”-shaped implant which may be folded to provide a cone-shaped or hemisphere-shaped implant depicted in FIG. 4D, useful in knee replacement procedures; FIG. 4E depicts a thin embodiment of the implant depicted according to FIG. 2, and FIG. 4F depicts the implant when it is folded onto itself to form a cone or hemisphere, useful in acetabular cup reconstruction and other procedures.

FIG. 5 provides representations of a number of further embodiments of the invention: FIG. 5A depicts an implant similar to that shown in FIGS. 2 and 4A, except that an asymmetric sector has been removed or excluded from the otherwise circular implant shape; FIG. 5B depicts the implant of FIG. 5A when folded upon itself to form a cone, or hemisphere, useful in acetabular cup and like reconstructions; FIG. 5C depicts a “donut”-shaped implant comprising a flat circular implant having a co-axial void, useful in acetabular cup reconstruction and like procedures where the implant is molded or press-fit to the void space; FIG. 5D depicts a hemi-shell shaped implant which may be press-fit into a bone void, such as in the acetabular cup; FIG. 5E depicts a cone-shaped or hemisphere-shaped implant which may be press-fit into a bone void, such as in the acetabular cup; FIG. 5F depicts a tube which, depending on diameter, may be press-fit or used in an impaction grafting procedure in a bone intramedullary canal; FIG. 5G depicts a nested pair of tubes or cones which may be used for repair of large femoral defects, optionally in association with impaction grafting procedures.

FIG. 6 provides representations of a number of further embodiments of the invention: FIG. 6A depicts a sheet while FIG. 6B depicts a strip for repair of traumatic fractures, for cranial and flat-bone repair applications, and for inter-transverse process fusions; FIG. 6C depicts a cord-shaped implant for wrapping or grouting of severe trauma defects, for spinal fusions, inter-transverse process fusions and the like; FIG. 6D depicts a wedge-shaped implant for tibial plateau repairs, joint fusions, and intervertebral body fusions; FIGS. 6E, 6F and 7 depict different embodiments of restrictive devices, useful in restricting cement or other flowable materials in plugged intramedullary canals and the like, as in femoral canals during impaction procedures; FIG. 6G depicts an ovoid or football shaped implant useful in repairing cystoid or like bone defects; FIG. 6H depicts a hemi-ovoid or hemi-football shaped implant useful in repairing cystoid or like bone defects; FIG. 6I depicts a spherical implant useful in repairing cystoid or like bone defects; FIG. 6J depicts a hemi-spherical implant useful in repairing cystoid or like bone defects.

FIG. 7 depicts an implant useful as a restrictive device for insertion into a canal, such as the intramedullary canal of a long bone, for example during a cementous impaction procedure.

FIGS. 8A-C provide X-ray evidence of the efficacy of an acetabular implant according to this invention.

FIGS. 9A-10 provide photomicrographs of the composition of this invention, before and after implantation.

FIGS. 10A-D provide further photomicrographs of the composition of this invention, before and after implantation.

FIGS. 11A-H provides a series of photographs and X-rays showing repair of a severe tibial complex compound fracture after removal of antibiotic loaded methacrylate beads and implantation of the composition according to this invention.

FIGS. 12A and 12B provide photographs of one embodiment of the implant according to this invention, and its moldability.

### SUMMARY OF THE INVENTION

This invention provides implants and methods for making and using the implants to repair a wide variety of orthopedic defects or lesions, including, for example, acetabular cup damage or repair procedures. The implant may be made from any of a number of known materials, by employing the specific shapes and methods provided herein. Alternatively, specific novel compositions disclosed herein may be used for this purpose. In one embodiment of this invention, the implant is placed in the acetabular socket or other defect requiring repair, and is molded to create a perfect fit between an overlay implant to be inserted into the acetabulum and the bone surface of the pelvis or other overlay implant and basal bony structure.

Accordingly, it is one object of this invention to provide a wide variety of desirably shaped implants for a wide variety of orthopedic applications.

It is another object of this invention to provide implant devices optimized in shape for repair of acetabular cup defects.

It is a further object of this invention to provide a preferred method for making a wide variety of desirably shaped implants useful in a wide variety of orthopedic applications.

It is a further object of this invention to provide a preferred method for repair of acetabular and other orthopedic defects.

It is yet a further object of this invention to provide desirably shaped implants which may be molded to create a perfect fit at the site of implantation.

Further still, it is another object of this invention to provide a dry, granular composition that is both osteoconductive and osteoinductive. The granular composition is preferably derived from autograft, allograft or xenograft tissue.

Another object of the subject invention pertains to a method of producing a dry, granular composition that is both osteoconductive and osteoinductive. Preferably, such method comprises mixing bone chips with and osteoinductive material to form a mixture, and drying the mixture such that the osteoinductive material adheres to the bone chips.

Further still, another object of the subject invention pertains to an osteogenic, cross-linked, composite implant, and methods of making and using same.

Other objects and advantages of this invention will become apparent from a review of the complete disclosure and the claims appended to this disclosure.

### DETAILED DESCRIPTION OF THE PREFERRED EMBODIMENTS

Any material having the following characteristics may be employed to produce a device having the shapes and utilities disclosed herein. However, it will be appreciated by those skilled in the art that acceptable implant materials having the shapes and utilities disclosed herein may be prepared even though one or more of the desired characteristics is absent. In preferred embodiments, the compositions used in accord with the teachings herein have one or more of the following characteristics:

a. The composition should be bioabsorbable.

b. The composition should be osteogenic.

c. The composition should be osteoinductive.

d. The composition should be osteoconductive.

e. The composition should be malleable or flexible prior to and shortly after implantation so that any desired shape may be produced.

f. The composition should be able to withstand freezing, freeze-drying or other methods of preservation and be able to withstand sterilization.

g. Upon implantation, the materials should fill voids and, if malleable prior to implantation, should then set-up as a hard material in the shape of the voids that have been filled.

Those skilled in the art will appreciate that any autograft, allograft or xenograft material that is molded, machined, cast or otherwise formed into the shapes for use according to this disclosure come within the scope of this invention. However, disclosed herein are specific compositions of preferred characteristics.

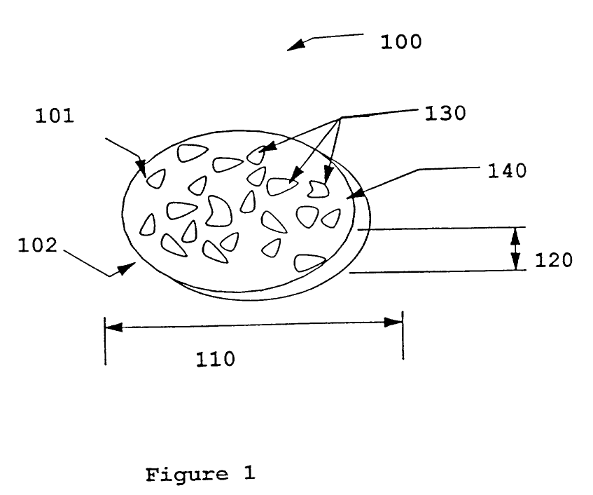

Referring now to FIG. 1, there is provided a representation of a first embodiment 100 of a device that may be prepared and used for acetabular implantation. The device 100 is substantially disk-shaped, having an upper surface 101, a lower surface 102, each of which is substantially circular, with a diameter 110. The diameter 110 is preferably in the range between about 35 and 55 mm, and most preferably is about 45 mm. The disk 100 has a height 120, which is preferably in the range between about 1 mm and about 10 mm, and is most preferably about 5 mm in height. Furthermore, the disk 100 may be composed of particulate matter 130 embedded or suspended in a base or carrier material 140. The particulate matter may be collagen sponge, cortical bone chips, cancellous bone chips, cortico-cancellous bone chips, hydroxyapatite or like ceramics, bioactive glass, growth factors, including but not limited to bone morphogenetic protein, PDGF, TGFβ, cartilage-derived morphogenetic proteins (CDMPs), vascular growth factors, and the like, demineralized bone, or any other material considered to be beneficial in the filling of bone or cartilaginous voids and the remodeling thereof into solid, healthy bone or cartilage through the processes of osseointegration (including osteogenesis, osteoinduction, or osteoconduction, as these terms are recognized in the art). The base or carrier material 140 may be any material, which retains a given form upon implantation into the void being filled behind an acetabular implant or in any other orthopedic application. Thus, for example, fibrin-containing compositions, which coagulate, maybe included in the carrier material 140, as may be various collagen formulations, hydroxylapatite, pleuronic polymers, natural or synthetic polymers, or carboxymethylcellulose, and combinations thereof. Preferably, the carrier material 140 comprises a sufficiently high concentration of gelatin, derived from human or animal tissue, or transgenic sources, such that prior to or upon implantation, the gelatin sets up to form a solid or semi-solid material of the desired shape. Use of gelatin as the base carrier material is considered desirable because, by simply heating a pre-formed device according to any of the embodiments of this invention, the implant device becomes flexible or malleable, and may be caused to precisely fit into the shape of any existing void or defect.

Where gelatin is employed as the base or carrier material, and cortical, cancellous or cortico-cancellous bone chips or demineralized bone is included in the carrier, the following percentages, on a weight basis, are considered desirable for formation of the variously shaped implants disclosed herein: the gelatin is preferably present at between about 12 to 27 weight percent. Demineralized bone is preferably present at between about 15 to 33 weight percent. Finally, cancellous bone chips, cortical bone chips or cortico-cancellous bone chips are preferably present at between about 70 to 100 volume percent. Alternatively, where a dry, granular composition is desired, the gelatin composition is preferably between about 2 to about 30 weight percent, and even more preferably between about 2 and 15 weight percent. The bone chips soak up the gelatin/demineralized bone material so that approximately equal volumes of the gelatin/demineralized bone and bone chips are preferably combined to produce the final preferred composition. Devices formed from this composition meet all of the requirements of a desirable implant material set forth above. Naturally, those skilled in the art will appreciate that a wide variety of supplemental constituents may be included in the composition. Thus, for example, growth factors, antibiotics, anti-inflammatory or other biologically active agents may be included at percentages that may be defined through routine experimentation, so long as the basic properties of the implant material is not adversely affected.

Using the appropriate concentration of gelatin, demineralized bone (to provide osteogenic factors) and cortical-cancellous bone chips (to provide structural strength and bone void filling capacity), a composition that is malleable above body temperature may be produced. Upon implantation or upon cooling, a solid device forms which may be machined or warmed for molding into any desired shape.

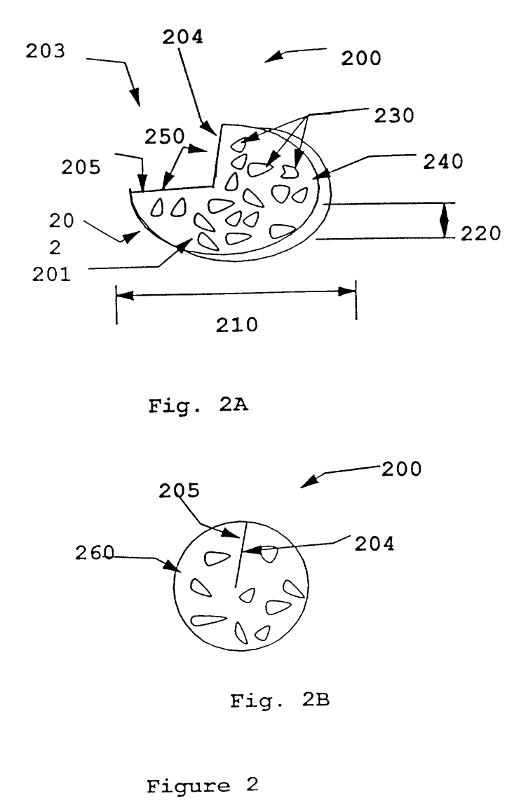

Referring now to FIG. 2A, there is shown a further embodiment 200 of the device according to this invention. This device is similar to that shown in FIG. 1, in that it has an upper surface 201, a lower surface 202, both of which are substantially circular. However, from this embodiment of the invention, a sector 203 has been removed or has not been included in the formation of the device, resulting in what will be referred to herein as a “filled-C-shape”. The purpose of this design modification is discussed in connection with the description of FIG. 2B below. The composition of the device shown in FIG. 2A and that of FIG. 1 may be similar, as are its desirable characteristics. The diameter 210 of the device 200 is preferably between about 50 mm and about 150 mm, and is most preferably between about 75 mm and 90 mm. The height 220 of the device is between about 1 mm and about 10 mm, and is most preferably about 5 mm. In addition, the particulate materials 230, when included, are similar to the particulate materials 130. The base or carrier material 240 is likewise similar to the carrier or base material 140. The angle formed between the adjacent sides 204 and 205 of the device 200 that exist by virtue of the absent sector 203 may be any angle greater than zero degrees and less than three-hundred and sixty degrees, and is preferably between about 90 and 150 degrees, and is most preferably about 120 degrees. In FIG. 2B, there is shown the device 200, wherein the adjacent sides 204 and 205 have been brought into contact, to form a substantially cone-shaped or hemisphere-shaped implant 260. Desirably, the device retains thermoplastic behavior for a limited amount of time after formation, so that the desired shape may be formed from the cone-shaped implant 260.

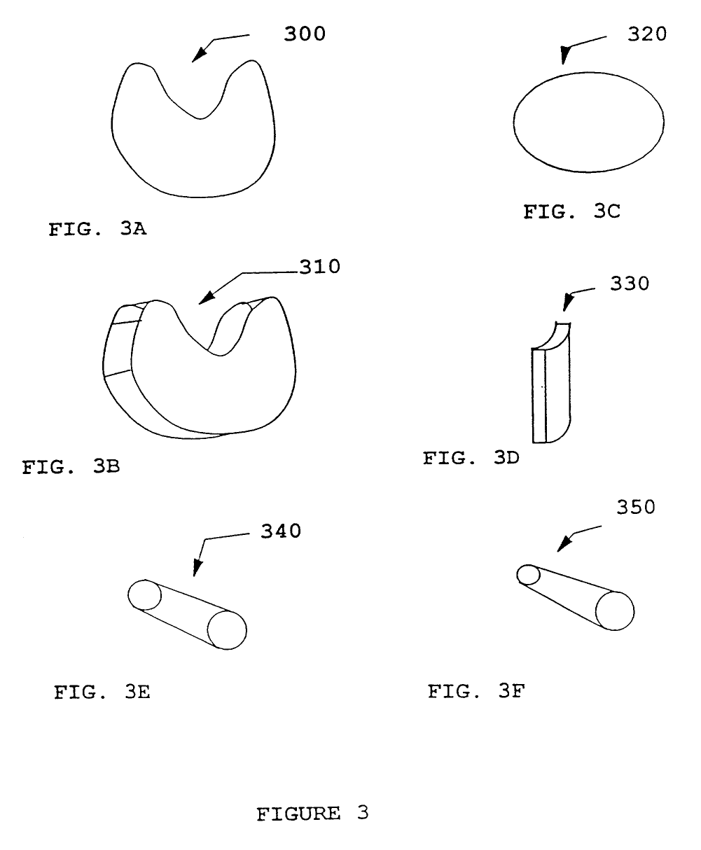

Based on the foregoing disclosure, it will be apparent to one skilled in the art that a wide variety of shapes and orthopedic applications may be addressed according to this invention. As examples of the wide-variety of applications and shapes that may be addressed by this invention, reference is made to FIGS. 3 through 7 included with this disclosure. Thus, FIG. 3 provides representations of a number of further embodiments of the invention: FIG. 3A depicts a thin “U”-shaped implant 300 useful in knee revision surgeries. FIG. 3B depicts a thicker “U”-shaped implant 310 useful in spinal fusion procedures. FIG. 3C depicts a thin oval implant 320 useful in knee revision and other surgical procedures. FIG. 3D depicts an implant shape 330 useful in posterior lumbar interbody fusion (“PLIF”) procedures. FIG. 3E depicts a dowel shaped implant 340, useful in spinal and joint fusions. FIG. 3F depicts a tapered dowel shaped implant 350, useful in spinal and joint fusions. According to the methods disclosed above, various percentages of particulate materials may be included in each of these disclosed shapes, as defined by routine experimentation, for particular applications. In addition, methods for conducting posterior lumbar interbody fusions, spinal fusions induced by dowels and the like may be carried out according to methods known in the art, but using the novel devices disclosed herein.

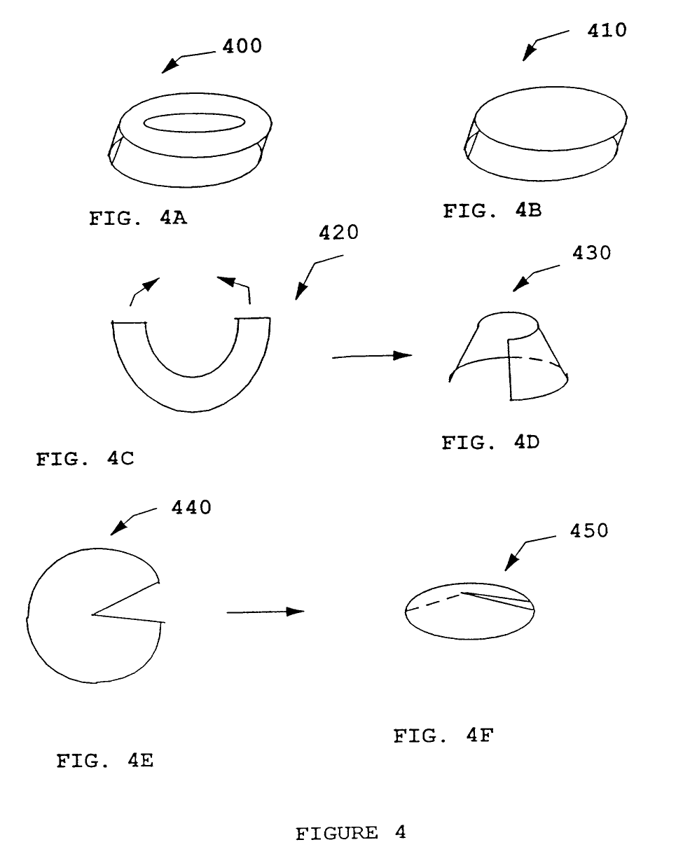

Further examples of implant shapes that may be produced and used according to the present disclosure are depicted in FIG. 4. Thus, FIG. 4A depicts a femoral or tibial ring shaped implant 400 useful in interbody fusion procedures. FIG. 4B depicts a round, plug-shaped implant 410 useful in cranial burr-hole repairs. FIG. 4C depicts a thin “U”-shaped implant 420 which may be folded to provide a cone-shaped or hemisphere-shaped implant 430 depicted in FIG. 4D, useful in knee replacement procedures. FIG. 4E depicts a thin embodiment 440 of the implant depicted according to FIG. 2, and FIG. 4F depicts the implant 450 when it is folded onto itself to form a cone, or hemisphere, useful in acetabular cup reconstruction and other procedures.

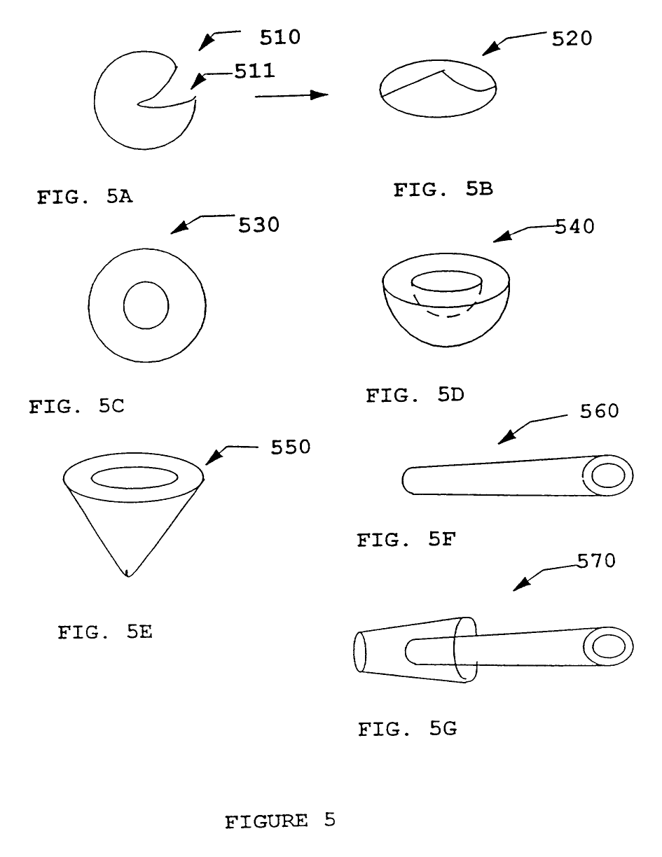

Additional examples of implant shapes that may be produced and used according to the present disclosure are depicted in FIG. 5. Thus, FIG. 5A depicts an implant 510 similar to that shown in FIGS. 2 and 4A, except that an asymmetric sector 511 has been removed or excluded from the otherwise circular implant shape. FIG. 5B depicts the implant of FIG. 5A when folded upon itself to form a cone or hemisphere 520, useful in acetabular cup and like reconstructions. FIG. 5C depicts a “donut”-shaped implant 530 comprising a flat circular implant having a co-axial void, useful in acetabular cup reconstruction and like procedures where the implant is molded or press-fit to the void space. FIG. 5D depicts a hemi-shell shaped implant 540 which may be press-fit into a bone void, such as in the acetabular cup. FIG. 5E depicts a cone-shaped or hemisphere-shaped implant 550, which may be press-fit into a bone void, such as in the acetabular cup. FIG. 5F depicts a tube 560 which, depending on diameter, may be press-fit or used in an impaction grafting procedure in a bone intramedullary canal. FIG. 5G depicts a nested pair of tubes or cones 570, which may be used for repair of large femoral defects, optionally in association with impaction grafting procedures. Each of these shapes may be fashioned by hand, molded, extruded or formed by other means known in the art. In addition, solid materials may be machined to produce the desired shapes, or because of the thermoplastic properties of gelatin, the desired shapes may be produced by known stereolithographic processes.

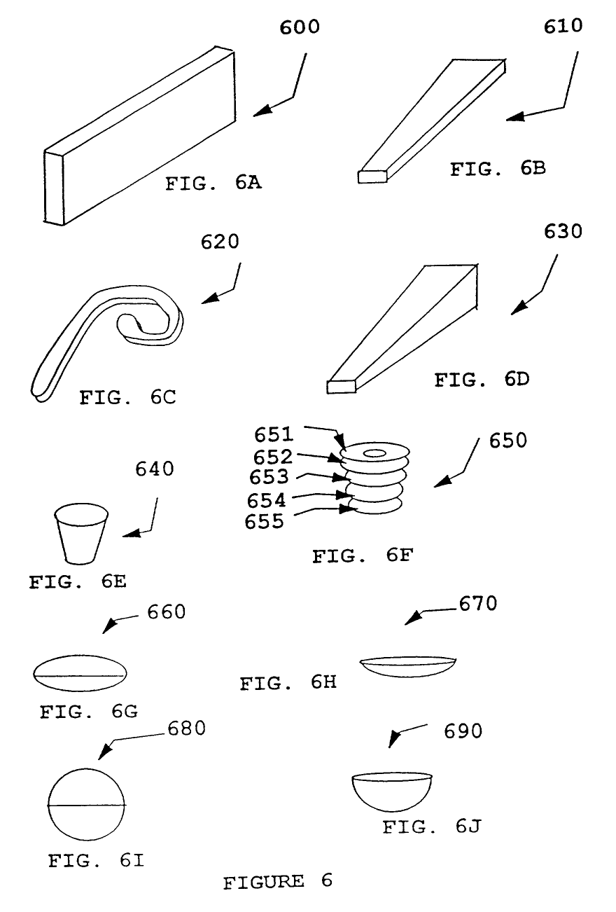

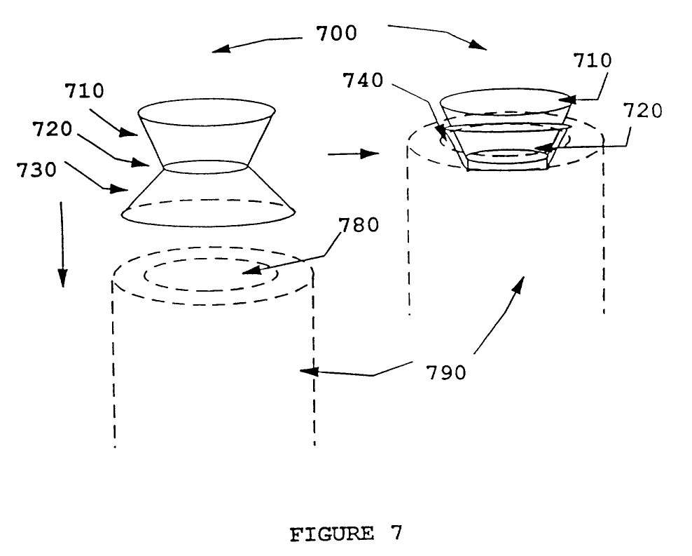

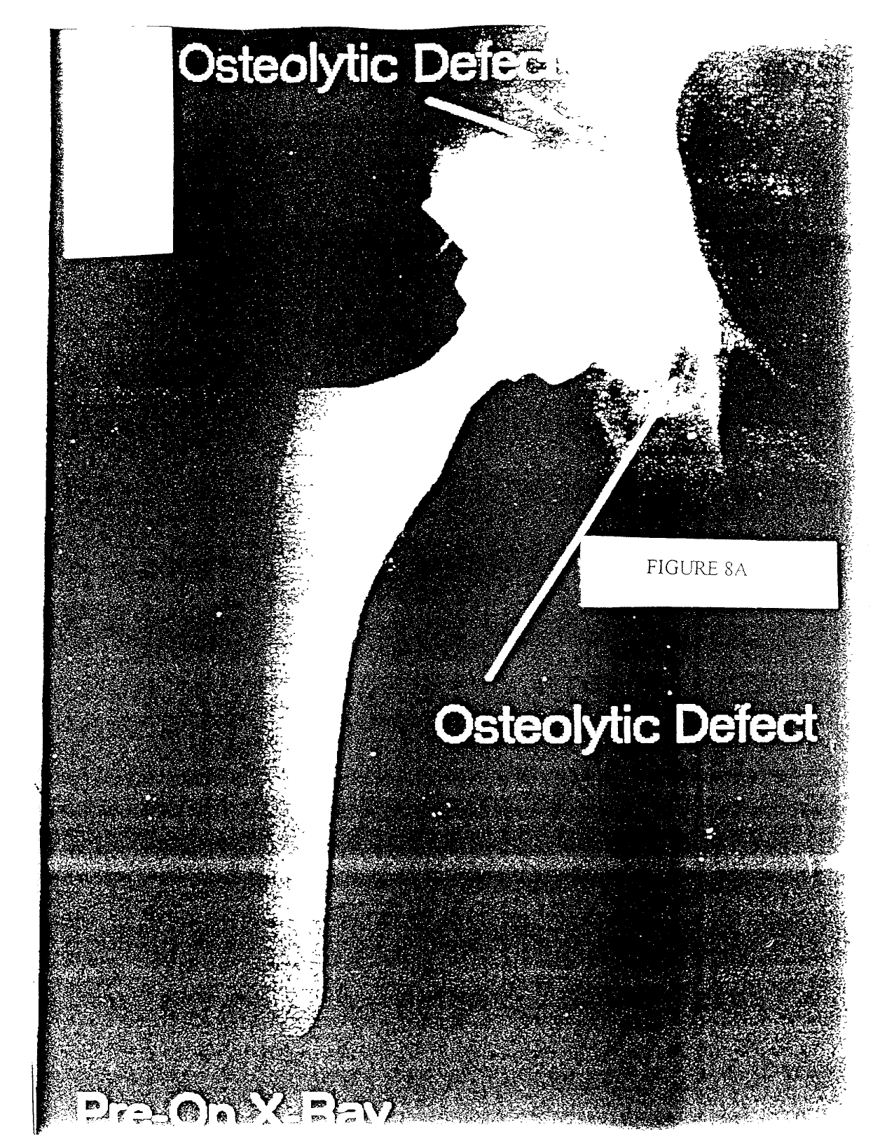

Yet further examples of the shapes that may be produced and used according to this invention are depicted in FIG. 6. Thus, FIG. 6A depicts a sheet 600 while FIG. 6B depicts a strip 610 for repair of traumatic fractures, for cranial and flat-bone repair applications, and for inter-transverse process fusions. FIG. 6C depicts a cord-shaped implant 620 for wrapping or grouting of severe trauma defects, for spinal fusions, inter-transverse process fusions and the like. FIG. 6D depicts a wedge-shaped implant 630 for tibial plateau repairs, joint fusions, and intervertebral body fusions; FIGS. 6E, 6F and 7 depict different embodiments of restrictive devices, 640, 650, 700, useful in restricting cement or other flowable materials in plugged intramedullary canals and the like, as in femoral canals during impaction procedures. The flow restrictor 640 has a classic “cork” stopper shape. The implant 650 has a tapered shape like that of the “cork” 640, but the device 650 is formed by a plurality of stacked “ribs” 651-655 of decreasing diameter. Naturally, the ribs may be formed by molding, such that separate elements 651-655 need to be separately produced. The implant 700 comprises an upper, solid portion 710 having a substantially “cork” shaped configuration. Affixed at seam 720 to the upper solid portion 710 is a thin, hollow, lower portion 730. The thin lower portion 730 folds upward about seam 720 upon insertion of the implant 700 into a lumen 780 of a bone 790 to form a tight seal 740 surrounding the upper plug portion 710. FIG. 6G depicts an ovoid or football shaped implant 660 useful in repairing cystoid or like bone defects. FIG. 6H depicts a hemi-ovoid or hemi-football shaped implant 670 useful in repairing cystoid or like bone defects. FIG. 6I depicts a spherical implant 680 useful in repairing cystoid or like bone defects. FIG. 6J depicts a hemi-spherical implant 690 useful in repairing cystoid or like bone defects.

Having generally described the invention, including the best mode and preferred embodiments thereof, the following section provides specific exemplary support for the invention as disclosed and claimed. However, the specifics of these examples are not to be considered as limiting on the general aspects of this invention as disclosed and claimed.

### EXAMPLE 1

### Repair of an Acetabular Cup Defect

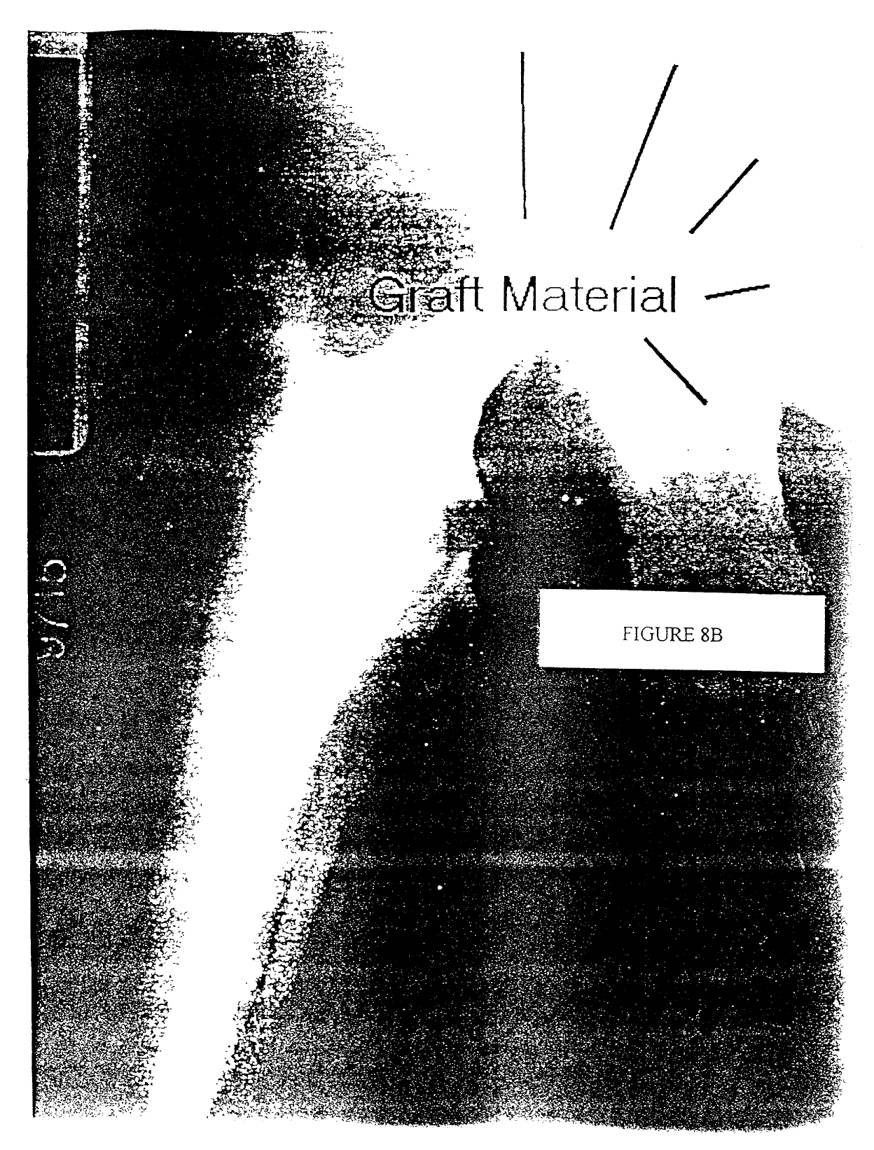

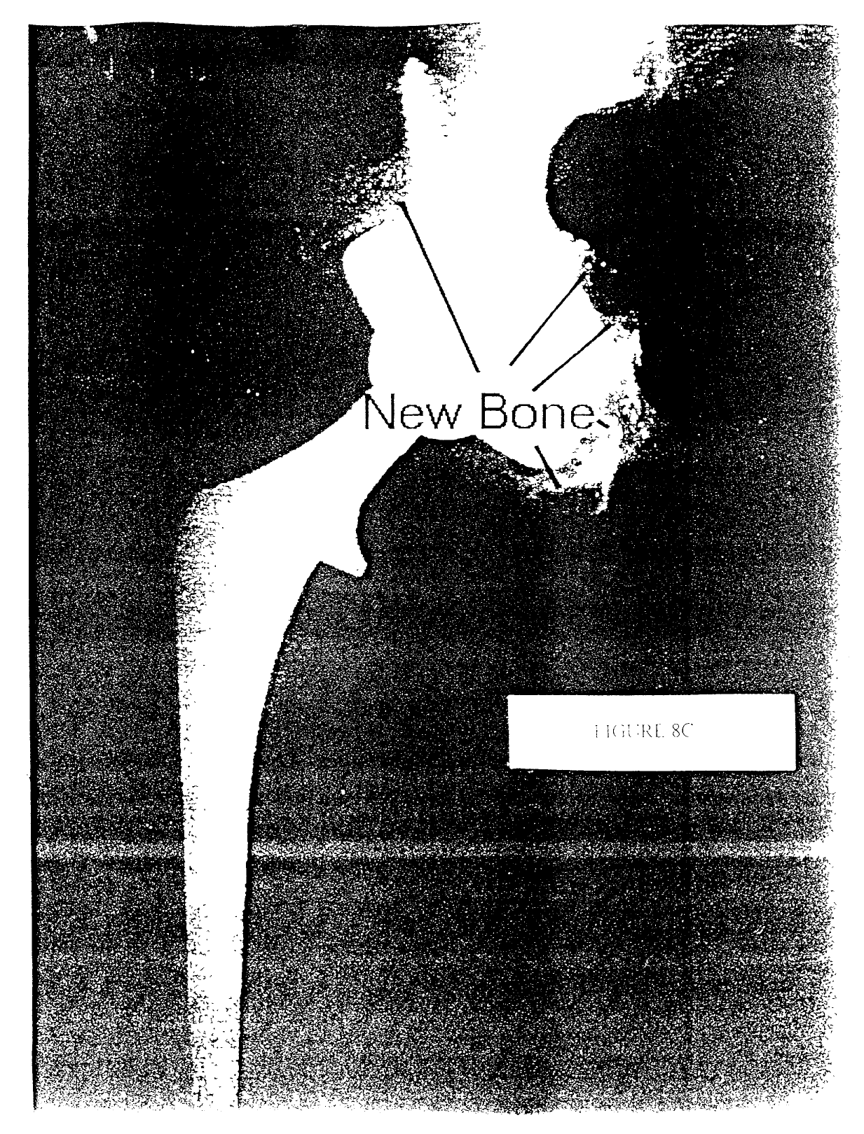

A patient presents with a severe osteolytic lesion behind a primary acetabular implant, due to wear-debris induced osteolysis. In this case, a revision surgery was indicated to replace the worn acetabular component and to remove the lesion. After removing the original acetabular component, the bone lesion was curetted out leaving a healthy bleeding bone mass. A cone- or hemisphere-shaped device was made from 100% v/v cortical-cancellous chips mixed with 68% v/v demineralized bone matrix in a gelatin carrier (24% w/w demineralized bone matrix, 26% w/w gelatin, 50% w/w water) was heated to soften the implant, which was then folded to form a cone or hemisphere. This softened cone or hemisphere of allograft was then forced into the curetted lesion and compressed with the fingers or a trial acetabular cup. A trial cup or a reamer was used to shape the allograft into the form of the back of the new acetabular component. Once the material hardened, the new acetabular component was placed on top of the allograft cup and screwed into place. The resulting efficacy is plainly evident in a series of X-rays of a patient that underwent this procedure. See FIG. 8.

FIG. 8A shows the pre-operative condition of an implant in which the osteolytic defect surrounding the implant articulating surface is clearly evident as the absence of bone mass in the X-ray. FIG. 8B shows an immediate post-operative X-ray, showing the implant with the above-described composition located where the osteolytic defect existed. FIG. 8C shows the same patient six months after completion of the osteolytic defect repair operation. Growth of new bone and repair of the defect is clearly evident.

### EXAMPLE 2

### Placement of a Primary Hip Acetabular Cup

Press-fit implants are used in younger patients because the long-term success of these implants is improved over those that are cemented into place using methacrylate bone cement. The reason for this improved long-term success is that the bone directly bonds to the surface of the implant. Because bone-to-implant bonding is improved by the incorporation of a porous coat in the implant, most press-fit orthopedic implants now have a porous coating. However, even with a porous coating, after explantation, most implants are found to only have bonded to the bone over approximately 20% of the surface area. Research has also shown that the long-term success of the implant is roughly correlated with degree of host-implant bonding. The degree of host-implant bonding is severely affected by the quality of the fit between the bone and the implant. If there is too much play in the bone-implant fit, then little or no bonding occurs and it will be necessary to cement the implant into place. By contrast, the osteoinductive, osteoconductive or osteogenic matrix according to this invention, which closely and concurrently interdigitates with both the porous surface of the implant and the bone into which the implant is inserted, facilitates repair of even poorly cut cavities in bone for press-fit insertion of implants. Interdigitation between the porous implant surface and bone causes bone to be induced or conducted from the bleeding bone into the porous coating and thereby induce much better bone-implant bonding. Bearing these considerations in mind, a young, otherwise healthy, patient presenting with osteoarthritis of the hip is treated as follows: It is noted that the degree of advancement of osteoarthritic bone destruction is such that drug-therapy is insufficient to relieve pain and the patient has limited mobility. In this case, a primary press-fit hip replacement is indicated. Through standard surgical techniques, the natural hip is removed and prepared for replacement with a metallic hip. The acetabulum is prepared by carefully reaming out a space that fits to the back of the acetabulum. A doughnut-shaped acetabular implant (FIGS. 4A or 5C) is prepared by warming in a water bath. The warm doughnut-shaped implant is placed into the patient's prepared acetabulum. While the doughnut-shaped implant is still warm, the porous acetabular cup is placed on top of the doughnut-shaped implant and is hammered into place. The particle size and viscosity of the doughnut-shaped implant material allows the material to easily flow into the porous coating of the implant and into the host's cancellous bone.

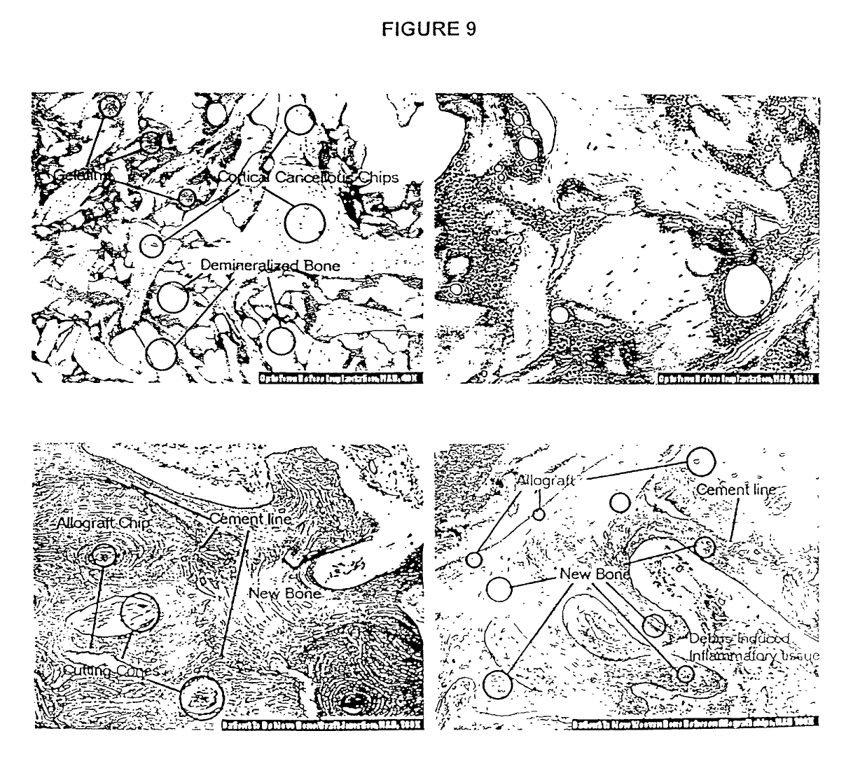

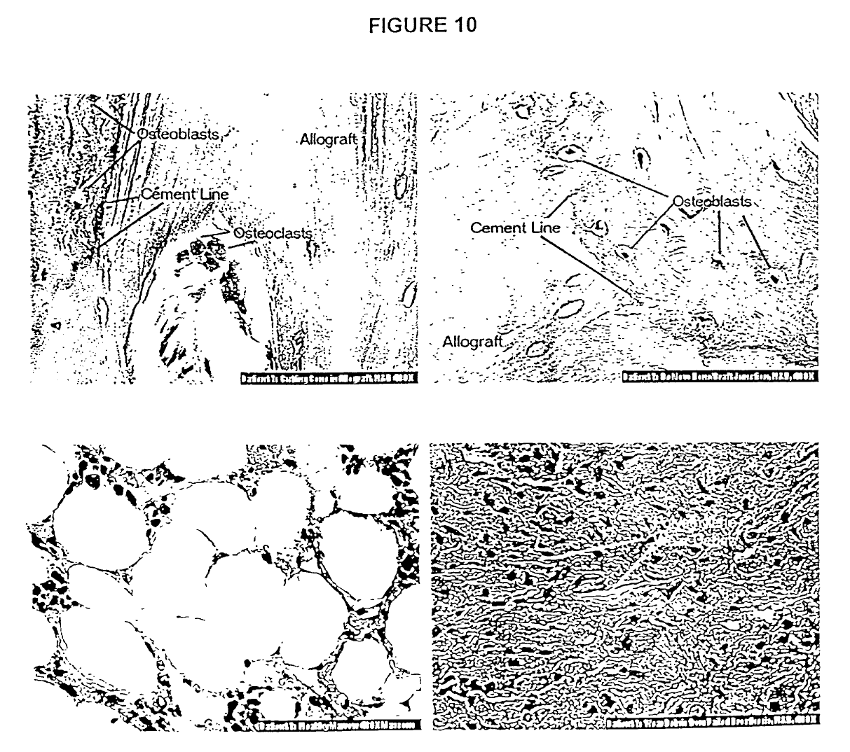

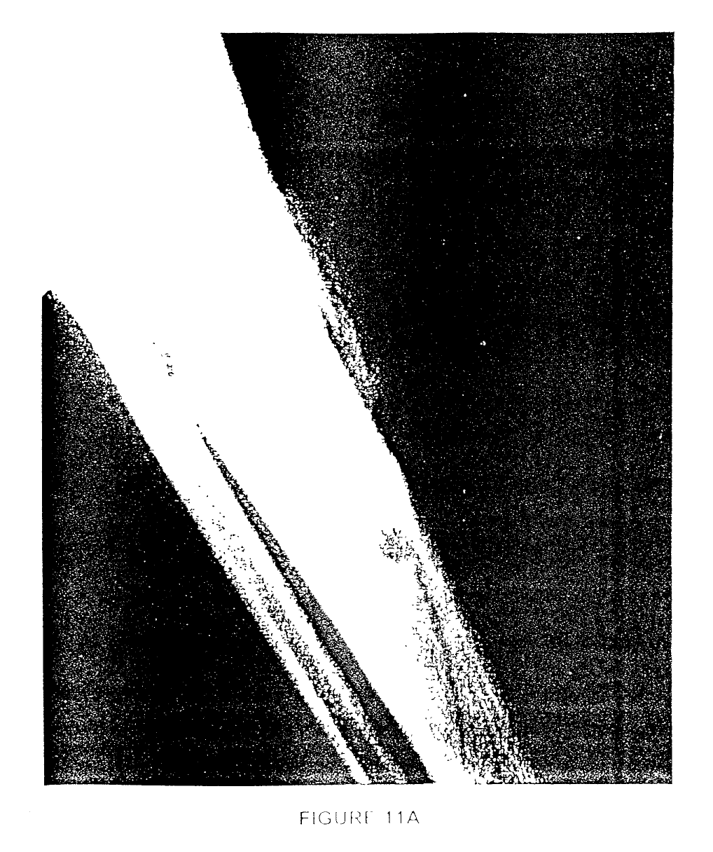

FIG. 9A shows a photomicrograph (40-X) of stained (H&E) composition according to this invention. Based on the staining, the different components of this composition are identified. Note the preferred relative uniformity, preferably between about 125 μm to about 5 mm, and preferably, between about 500 μm to about 1 mm or between about 1 mm to about 3.35 mm. We have found that bone chips uniformly formed within these preferred size ranges result in surprisingly improved induction and conduction of new bone formation and improved handling of the composition. In FIG. 9B, the same material is viewed under higher magnification (100X), showing the interpenetration of gelatin into and onto the cortical-cancellous chips and demineralized bone matrix of the composition. FIG. 9C shows a biopsy after implantation of this composition in a human female, 6 months after implantation, showing new bone formed onto the surface of a piece of allograft (H&E, 100X). Noticeable are the numerous cutting cones within the mineralized allograft, indicating that the allograft bone will continue to be fully remodeled over time. FIG. 9D shows a biopsy of new woven bone between mineralized allograft chips (H&E, 100X). It should be noted that the area between the spicules would normally be filled with healthy marrow. However, in this case, it can be seen that these areas are filled with fibrous inflammatory tissue cause by wear debris from a failed prosthesis. FIG. 10A shows additional photomicrographs of a biopsy from a human female six months after implantation of the composition of this invention. This photograph shows details of a cutting cone in a piece of mineralized allograft (H&E, 400X), revealing the presence of osteoclasts, osteoblasts and a cement line, whereby implant material is remodeled into normal healthy recipient bone. FIG. 10B shows a detailed photomicrograph of a cement line between mineralized allograft and new bone (H&E, 400X), revealing osteoblasts at the periphery of the allograft. FIG. 10C is a photomicrograph of normal marrow found in areas adjacent newly formed bone, unaffected by wear debris (H&E, 400X). FIG. 10D provides a detail of the filamentous wear debris found in the fibrous inflammatory tissue (H&E, 400X).

These photomicrographs clearly demonstrate that the composition of this invention, whether provided in a pre-formed shape, or molded to fit precisely into a recipient implant site, results in rapid remodeling and osteoinductive and osteoconductive effects. Accordingly, gaps that might otherwise prevent new bone formation and ingrowth may be filled with the composition of this invention to induce union between bone and implant materials. Thus, in one specific embodiment of this invention, a porous implant or an implant having a porous coating is contacted with the composition according to this invention. For example, in a total knee arthroplasty, typically an implant having 500-700 μm metal beads contacted with the sawn-off end of the femur. By application of the composition of this invention at the union surface, rapid ingrowth of bone into the metal bead interstices is induced by driving the implant surface into a pre-formed or molded shape formed from the composition according to this invention.

### EXAMPLE 3

### Repair of a Complex Compression Fracture

Complex compression fractures are frequently associated with significant bone loss because the nature of the fracture is such that the bone is shattered and many of the bone fragments are irretrievable. Current practice dictates the collection of as many bone pieces as possible and the placement of those pieces back into the fracture site. Missing pieces are normally replaced with morselized autograft taken from the hip, from the rib, or from the fibula. Occasionally, artificial grafting materials are used with limited success. Allografts have also been used, with varying success, largely dependent upon the nature of the allograft and its source. The application of malleable or moldable pre-formed and appropriately-shaped implants to this type of repair allows the surgeon to effectively replace the lost bone, without inducing additional trauma by harvesting autograft from another surgical site.

Accordingly, a complex fracture, such as one in the radius, is repaired by following standard surgical techniques to clean the fracture site followed by placement within the fracture of malleable allograft implant material of this invention in the form of a football, sphere, hemi-football, hemisphere, or sheet/strip. Shattered bone particles are packed around the malleable material. Alternatively, the shattered particles of bone are placed into the fracture site and then strips or cords of malleable implant material according to this invention are laid over the fracture site. Malleable cord-shaped implant material of this invention is optionally used as an adjunct or in place of circlage wires to fix the fracture fragments into place.

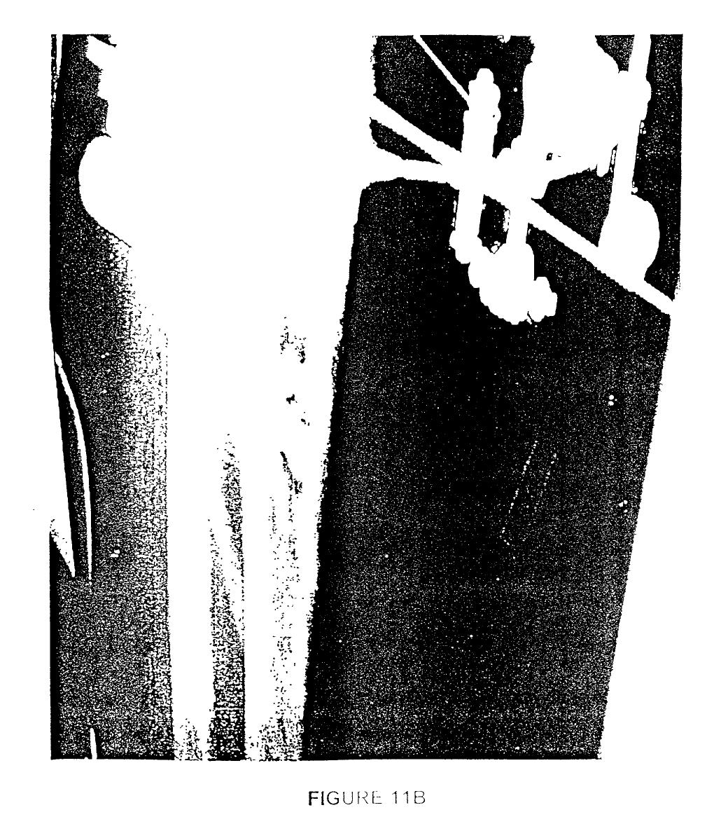

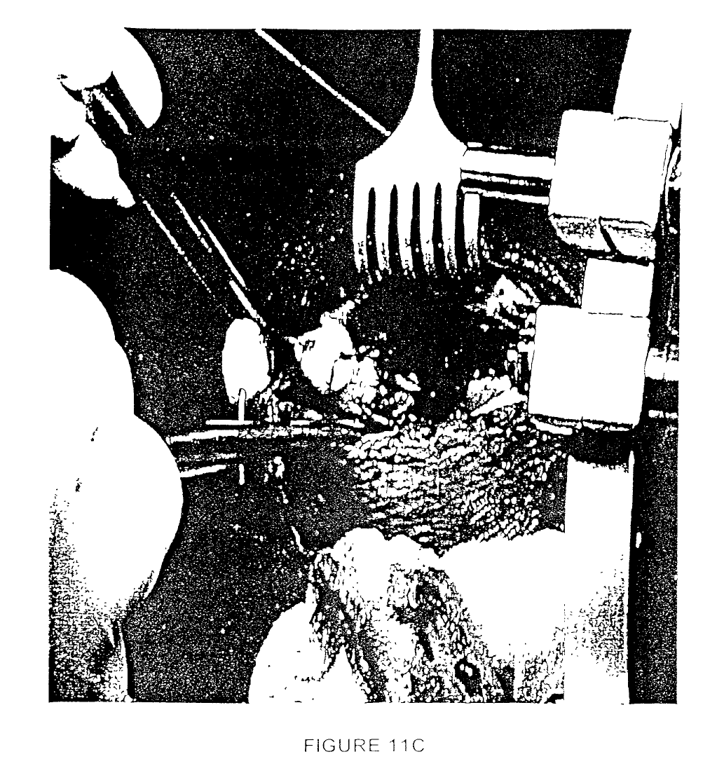

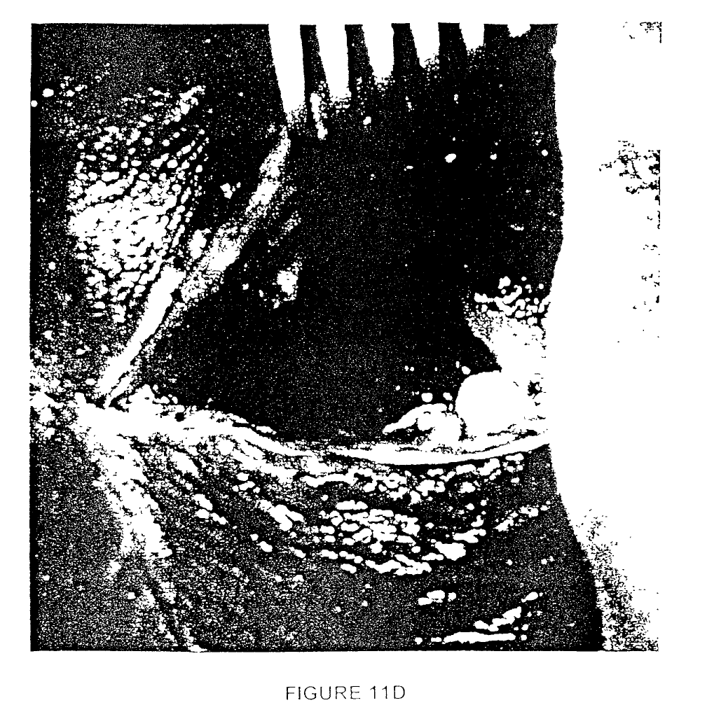

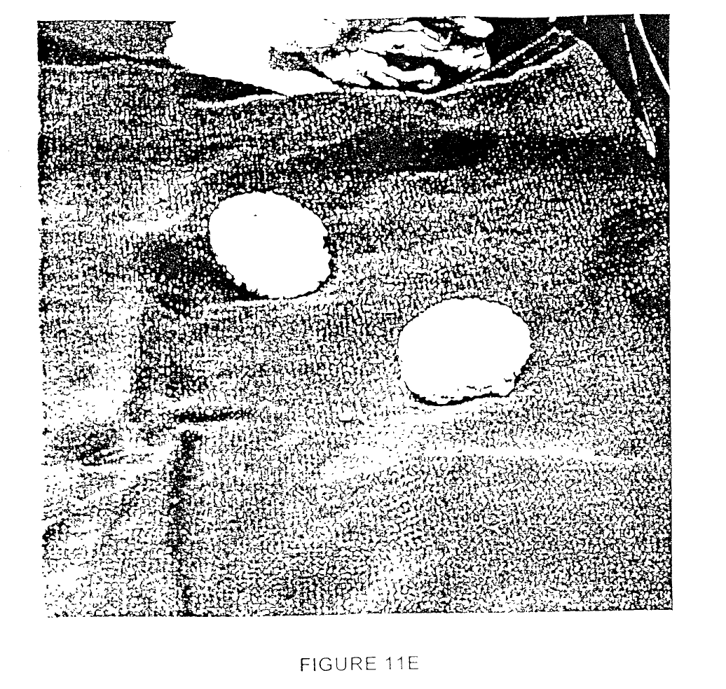

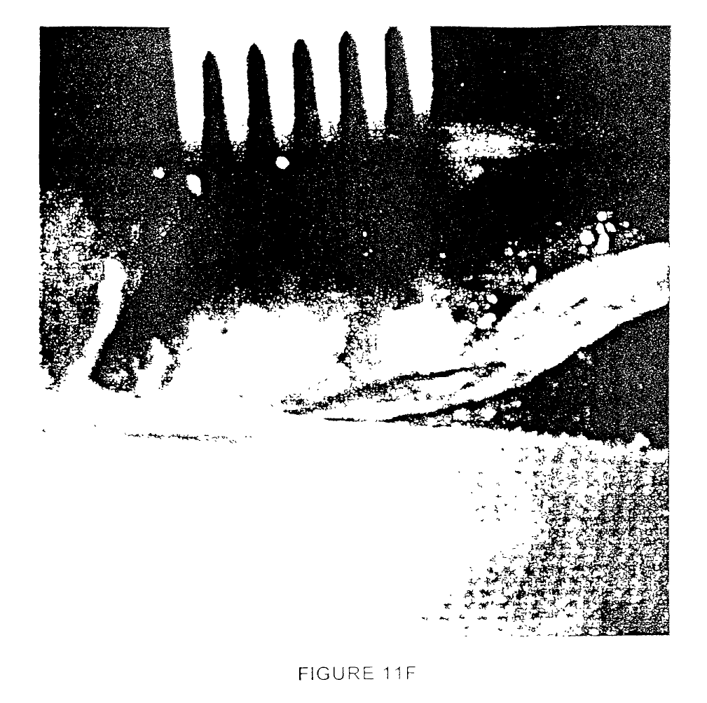

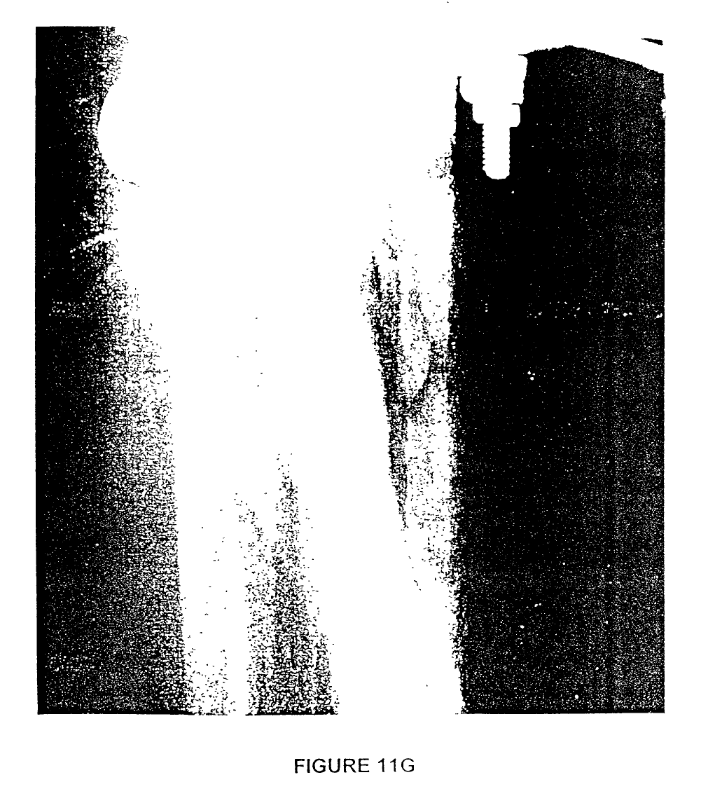

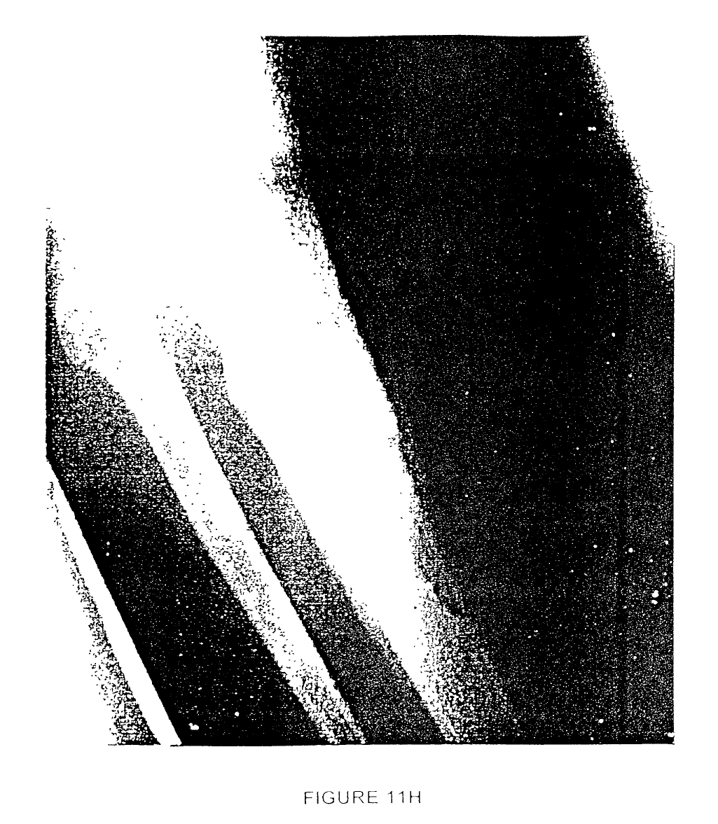

FIG. 11 shows a surgical procedure in a tibia of a patient who experienced a complex compound fracture into which, for a period of four weeks, had been implanted gentamycin impregnated polymethylmethacrylate “beads on a string”. FIG. 11A shows circular structures in the center of the photograph which are the beads, implanted in an effort to treat a local infection at a fracture site. FIG. 11B shows a pre-operative X-ray of the surgical set-up, again with the implanted beads visible in the bone void. FIG. 11C shows the intra-operative procedure whereby the implanted beads were removed. FIG. 11D shows the large cavity remaining after removal of the beads. FIG. 11E shows a photograph of the composition according to this invention, formed in the shape of two dry eight cubic centimeter disks, prior to implantation. FIG. 11F is an intra-operative photograph, after implantation of sixteen cubic centimeters of the composition of this invention. The implant material is clearly visible, and as can be seen from this photograph, is moistened by body fluids, but is not soluble and is not washed away. FIG. 11G shows the implant site immediately post-implantation. The site of the implant within the void can be discerned as a faint cloud within the void. FIG. 11H is an X-ray photograph of the implant site six-weeks post implantation. It can clearly be seen that the implant material has remodeled to form solid bone mass, while a portion of the void into which implant material was not or could not be implanted remains a void.

### EXAMPLE 4

### Repair of Osteolytic Cysts

Osteolytic cysts and other growths on bone that must be removed are typically difficult to replace. Traditional practice dictates that large cystic defects be filled with weight-bearing allograft or autograft. Alternative techniques have employed synthetic materials with limited success.

In this application of the malleable implant material of this invention, cystic defects are repaired after removal of the cyst by placing warm, malleable implant material according to this invention onto the defect and forming it to completely fill the void. The material according to a preferred embodiment of this invention remodels into natural bone in a period ranging from between about 6 weeks to about 9 months.

### EXAMPLE 5

### Intertransverse Process Spinal Fusion

Intertransverse process spinal fusion is generally accomplished by the joint application of both metallic fixation devices and the use of autograft, which is generally harvested from the patient's hip. The autograft harvest is associated with a high rate of morbidity (21%).

The use of a grafting material that is effective without the necessity of harvesting autograft would greatly benefit patients in need of such procedures. Accordingly, after standard surgical preparation including rigorous decortication of the transverse processes and the facets of two adjoining vertebrae, a malleable pre-molded form (strips or cords) of the malleable implant material of this invention are lain gutter alongside the vertebral bodies. Local bone reamings are optionally mixed or intermingled with the still warm and malleable implant material and then the implant material is pressed into the bleeding bone bed.

### EXAMPLE 6

### Filling of Cranial Burr Holes

Cranial burr-holes are created whenever it is necessary to cut into the skull in order to gain access to the brain. Current technique dictates the use of plaster of paris-like substances, metallic meshes, and bone waxes to fill these holes, or to not fill them at all. None of the commonly employed products and procedures induce bone to grow across the defect, and some of these products and procedures actually inhibit the growth of the bone.

Accordingly, in this application, a disk-shaped piece of pre-molded implant material according to this invention is placed, warm, into the burr-hole defect, with a small lip of the implant material remaining above the surface to serve as a temporary support for the material. It is anticipated that the temporary support is unnecessary after a period of several days, after which the plug is expected to remain in place on its own. It is anticipated that new bone grows into the remaining gap to completely bridge the gap within about 6 weeks to about 9 months.

### EXAMPLE 7

### Molding of the Composition of this Invention

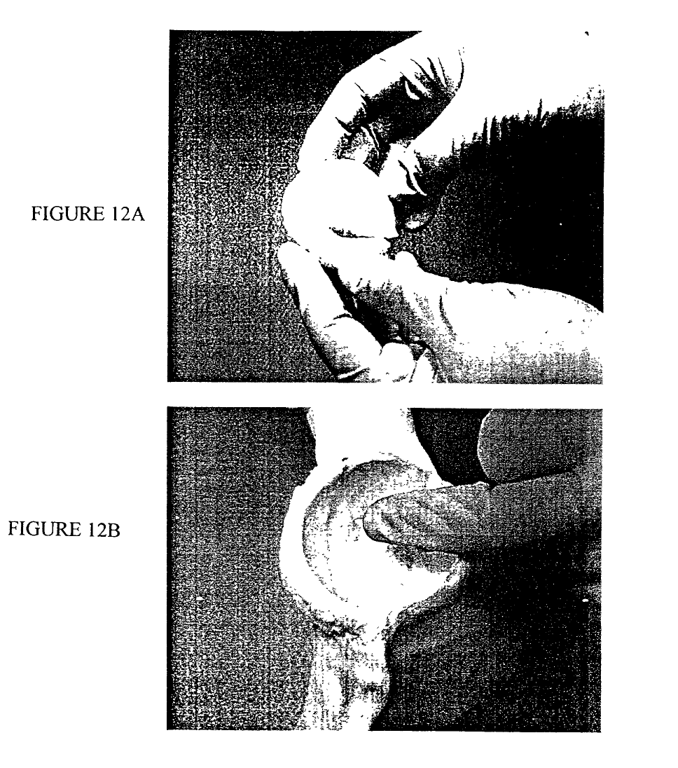

FIG. 12 shows the formability and moldability of the composition of this invention. FIG. 12A shows a dry cone or hemisphere of the composition. Upon hydration and heating to about 43 to about 49 degrees centigrade, the material becomes moldable, and re-sets at body temperature, as shown in FIG. 12B, where the moldable material is being press-fit by finger pressure into a cavity. Once set-up, the material is easily reamed or drilled for placement of any desired prosthesis.

### EXAMPLE 8

### Production of Cortical, Cancellous or Cortical-Cancellous Bone Chips for Inclusion in the Composition of this Invention

Corticocancellous chips were processed from allograft obtained from the iliac crest, iliac crest segments and from metaphyseal cancellous bone. When metaphyseal ends and iliac crests are used, an approximate mixture of 20%:80% to about 50%:50% cortical:cancellous bone chips is obtained. The bone chips are produced after debridement and antimicrobial treatment in a class 10 or class 100 cleanroom. Appropriately cleaned and sectioned bone was ground in a bone mill fitted with a sieve, to ensure that all collected bone chips are of a fairly uniform size between about 125 μm and about 5 mm. Preferably, the collected bone chips are in the size range of about 125 μm to about 1 mm or between about 1 mm and 3.35 mm. The ground bone chips were soaked in peroxide, with sonic treatment. The peroxide treatment was repeated until no more fat or blood was visible, the peroxide was decanted and the chips were soaked in povidone iodine solution. The chips were then rinsed with water, and then soaked in an ascorbic acid solution, followed by treatment with isopropanol, with sonic treatment. Finally, the chips were treated with a further peroxide soak, followed by a water rinse, and then lyophilization. The dried chips were then sieved to select the desired size range of bone chips desired. Samples were cultured to ensure sterility.

### EXAMPLE 9

### Preparation of the Composition of this Invention for Molding into Desired Shapes

A known weight of ground lyophilized gelatin of up to 850 μm particle size was mixed with a known weight of demineralized bone particles of between about 250 μm and 850 μm. A known weight of water was added to the combined gelatin and demineralized bone, and thoroughly mixed. The gelatin, water, demineralized bone composition was then warmed to form a paste of known volume, and a fifty-percent to 100 percent volume of corticocancellous bone chips of between about 125 μm and 5 mm particle size was then added and the entire composition was thoroughly mixed, with repeated warming steps as needed to ensure thorough mixing. The mixed composition was then molded into desired shapes, which are stored in sealed sterile pouches or like containers. Upon use, a surgeon uses the shaped material in its pre-formed shape, or warms the material until it becomes moldable, before implanting the material into a desired implant site.

### EXAMPLE 10

### Production of a Dry, Granular, Graft Composition

Impaction grafting is typically used to fill voids in long bones resulting from the removal of a failed prosthesis. In most cases, these failed prostheses are removed because they become loose, which results in significant bone loss and enlargement of the intramedullary canal. To help support a new replacement prosthesis, the intramedullary canal is packed with suitable materials during revision surgery (see U.S. Pat. No. 6,045,555). Recently, it has been found to be desirous to use dry, granular materials to replenish the loss of bone and to provide support for the replacement prosthesis, as they have been found to pack better and are able to be delivered deep into bone defects in a more uniform fashion. Presently non-inductive, cortical-cancellous chips are used in impaction grafting techniques for total joint revisions to provide an osteoconductive scaffold to allow bone to regenerate. Remodeling of the implanted chips can be a slow process because this type of allograft regenerates through a process of “creeping substitution”.

One embodiment of the subject invention alleviates the problems of current materials by providing a granular bone material that comprises bone chips that have an osteoinductive material adhered thereto. Specifically exemplified are bone chips (cortical, cancellous, or cortical-cancellous) that have demineralized bone matrix (DBM) adhered to their outer surface. The osteoinductive bone chips of the subject invention provide significant advantages over current impaction grafting materials, such as increased rates and amounts of bone remodeling. Those skilled in the art will appreciate many other uses of the subject osteoinductive bone chips, in addition to their importance in impaction grafting techniques.

The subject osteoinductive bone chips can be made, for example, by mixing bone chips (such as those produced per Example 8 above), gelatin, DBM, and water together to form a slurry. Once thoroughly mixed, the slurry is then freeze dried according to conventional methods, whereby upon drying, the gelatin and DBM adhere to the bone chips. After drying a porous cake is formed, which is then broken up by conventional means such as a mortar and pestle. Those skilled in the art will appreciate that many other osteoinductive substances besides DBM can be used in accord with the principles of this embodiment, such as, e.g., osteoinductive growth factors. Furthermore, while gelatin may be a preferred carrier material, skilled artisans will appreciate that other carrier materials can be substituted for, or added to, gelatin, such as, e.g., fibrin-containing compositions, collagen compositions, pleuronic polymers, natural or synthetic polymers, cellulose derivatives such as carboxymethylcellulose, hyaluranic acid, chitin, or combinations of the foregoing.

In one example, 100 cc of cortical-cancellous chips were combined with 30 cc of DBM and 20 cc of a 3% gelatin (275 Bloom, Dynagel, lot # 13005) mixture. The ingredients were mixed thoroughly by conventional means and then lyophilized.

In another example, 60 cc of a 5% gelatin (275 Bloom) mixture was combined with 60 cc of DBM and thoroughly mixed. After mixing, 240 cc of cortical-cancellous chips were added to the gelatin/DBM mixture and the gelatin/DBM/CCC combination was kneaded to form a dough-like mixture. The gelatin/DBM/CCC combination was then spread into a thin sheet on a stainless steel container. 200 cc of a 3% gelatin mixture was applied to the gelatin/DBM/CCC combination. The gelatin/DBM/CCC combination was then lyophilized. Lyophilization of the gelatin/DBM/CCC combination formed a cake that was broken up and sifted through a 5.6 mm sift.

### EXAMPLE 11

### Cross-Linked Implant Having Increase Structural Integrity

In a further embodiment, the subject invention pertains to an implant made by molding bone particles (cortical, cancellous, and/or corticocancellous bone chips) into predefined shapes. Prior, subsequent and/or during the molding of these particles, the particles are cross-linked using conventional cross-linking methods known in the art, such as by glutaraldehyde treatment or other chemical treatments, dihydrothermal treatment, enzymatic treatment, or irradiation (e.g., gamma, ultraviolet or microwave). The particles used to produce the cross-linked implant are fully mineralized, partially demineralized, or fully demineralized, or alternatively comprise a combination of mineralized and demineralized particles. In view of the teachings herein, those skilled in the art will appreciate that the mechanical properties of this embodiment can be controlled by the extent of demineralization of the particles before cross-linking, or demineralizing (fully, partially, or segmentally) the resultant molded implant.

Constructing whole implants with a mold, or parts of an implant that can be subsequently assembled, would enable a wide array of different shapes having simple or very complex geometries. Examples of shapes for this embodiment include, but are not limited to, a sheet, plate, disk, cone, suture anchor, pin, wedge, cylinder, screw, tube or lumen, or dowel. As mentioned above, in addition to molding, a basic shape can be formed whereby the implant can be machined using conventional bone machining techniques.

Typical chemical cross-linking agents used in accord with this embodiment include those that contain bifunctional or multifunctional reactive groups, and which preferably react with surface exposed collagen of adjacent bone particles. By reacting with multiple functional groups on the same or different collagen molecules, the chemical cross-linking agent increases the mechanical strength of the implant.

The cross-linking step of the subject embodiment involves treatment of the bone particles and/or additional binder substance to a treatment sufficient to effectuate chemical linkages between adjacent molecules. Typically, such linkages are between adjacent collagen molecules exposed on the surface of the bone particles. Naturally, chemical linkages can also occur between adjacent molecules of the binder substance, or between the molecules of the binder substance and of the bone particles. Crosslinking conditions include an appropriate pH and temperature, and times ranging from minutes to days, depending upon the level of crosslinking desired, and the activity of the chemical crosslinking agent. Preferably, the implant is then washed to remove all leachable traces of the chemical.

Suitable chemical crosslinking agents include mono- and dialdehydes, including glutaraldehyde and formaldehyde; polyepoxy compounds such as glycerol polyglycidyl ethers, polyethylene glycol diglycidyl ethers and other polyepoxy and diepoxy glycidyl ethers; tanning agents including polyvalent metallic oxides such as titanium dioxide, chromium dioxide, aluminum dioxide, zirconium salt, as well as organic tannins and other phenolic oxides derived from plants; chemicals for esterification or carboxyl groups followed by reaction with hydrazide to form activated acyl azide functionalities in the collagen; dicyclohexyl carbodiimide and its derivatives as well as heterobifunctional crosslinking agents; hexamethylene diisocyante; sugars, including glucose, will also crosslink collagen.

It is known that certain chemical cross-linking agents, e.g., glutaraldehyde, have a propensity to exceed desired calcification of cross-linked, implanted biomaterials. In order to control this calcification, certain agents can be added into the composition of the subject embodiment, such as dimethyl sulfoxide (DMSO), surfactants, diphosphonates, aminooleic acid, and metallic ions, for example ions of iron and aluminum. The concentrations of these calcification-tempering agents can be determined y routine experimentation by those skilled in the art.

When enzymatic treatment is employed, useful enzymes include those known in the art which are capable of catalyzing crosslinking reactions on proteins or peptides, preferably collagen molecules, e.g., transglutaminase as described in Jurgensen et al., The Journal of Bone and Joint Surgery, 79-a(2), 185-193 (1997), herein incorporated by reference.

Formation of chemical linkages can also be accomplished by the application of energy. One way to form chemical linkages by application of energy is to use methods known to form highly reactive oxygen ions generated from atmospheric gas, which in turn, promote oxygen crosslinks between surface-exposed collagen. Such methods include using energy in the form of ultraviolet light, microwave energy and the like. Another method utilizing the application of energy is a process known as dye-mediated photo-oxidation in which a chemical dye under the action of visible light is used to crosslink surface-exposed collagen.

Another method for the formation of chemical linkages is by dehydrothermal treatment which uses combined heat and the slow removal of water, preferably under vacuum, to achieve crosslinking of bone particles. The process involves chemically combining a hydroxy group from a functional group of one collagen molecule and a hydrogen ion from a functional group of another collagen molecule reacting to form water which is then removed resulting in the formation of a bond between the collagen molecules.

The bone particles employed in the composition can be powdered bone particles possessing a wide range of particle sizes ranging from relatively fine powders to coarse grains and even larger chips. Thus, e.g., powdered bone particles can range in average particle size from about 0.05 to about 1.2 cm and preferably from about 0.1 to about 1 cm and possess an average median length to median thickness ratio of from about 1:1 to about 3:1. If desired, powdered bone particles can be graded into different sizes to reduce or eliminate any less desirable size(s) of particles which may be present.

In a preferred variation of this embodiment, particles of demineralized bone matrix are mixed with a predetermined volume of a buffered formalin solution, and the resulting mixture is placed into a mold in the shape of a screw. The mixture is retained in the mold for 48 hours and the cast is removed and allowed to dry for an additional 24 hours.

Prior, during or subsequent to subjecting the bone particle composition to a cross-linking treatment, an amount of pressure can be applied to the composition. Application of pressure can aid in the formation and integrity of the implant. However, one advantage of the subject cross-linked embodiment is that it provides an implant with a porous structure which encourages the revascularization of the implant, and provides an architecture that encourages the migration and attachment of progenitor cells into the implant. Naturally, application of high pressure to the implant decreases the porosity of the implant, and should be avoided when porosity of the implant is needed for the specific application. Furthermore, another advantage of the subject embodiment is that it allows for production of implants having irregular and/or complex structures. These complex structures are preferably produced by making predefined molds into which the bone particle composition is disposed and allowed to set. Application of pressure would in most instances be counterproductive in producing such complex structures. Nevertheless, it is recognized that slight pressures may be applied during the formation of pre-selected shapes for the subject embodiment. Preferably, slight pressures for these purposes relate to about 975 psi or less. More preferably, slight pressures relate to between about 0 psi and about 500 psi.

The teachings of all the references cited throughout this specification are incorporated by reference to the extent they are not inconsistent with the teachings herein.

What is claimed is:

1. A method of producing an osteogenic, composite implant comprising the steps of:
obtaining a composition of bone particles, wherein said bone particles comprise fully mineralized bone particles, partially or fully demineralized bone particles, or a combination thereof;
forming said composition into a predetermined shape; and
subjecting said composition to a cross-linking treatment.

2. The method of claim 1, wherein said bone particles are partially or fully demineralized.

3. The method of claim 1, wherein said cross-linking treatment comprises contacting said bone composition with a chemical agent selected from the group consisting of mono- and di-aldehydes, including glutaraldehyde and formaldehyde; polyepoxy compounds such as glycerol polyglycidyl ethers, polyethylene glycol diglycidyl ethers and other polyepoxy and diepoxy glycidyl ethers; tanning agents including polyvalent metallic oxides such as titanium dioxide, chromium dioxide, aluminum dioxide, zirconium salt, as well as organic tannins and other phenolic oxides derived from plants; chemicals for esterification or carboxyl groups followed by reaction with hydrazide to form activated acyl azide functionalities in the collagen; dicyclohexyl carbodiimide and its derivatives as well as heterobifunctional crosslinking agents; hexamethylene diisocyante; and sugars such as glucose.

4. The method of claim 1, wherein said cross-linking treatment comprises contacting said composition with an enzyme.

5. The method of claim 4, wherein said enzyme is transglutiminase.

6. The method of claim 1, wherein said cross-linking treatment comprises dihydrothermal treatment of said composition.

7. The method of claim 1, wherein said cross-linking treatment comprises irradiation of said composition.

8. The method of claim 1, wherein said composition further comprises a binding agent selected from the group consisting of collagen, gelatin, fibrinogen, thrombin, elastin, albumin, keratin, chitin, gelatin-resorcinol-formaldehyde glues; collagen-based glues; cellolosics such as ethyl cellulose; bioaborbale polymers such as starches, polylactic acid, polyglycolic acid, polylatic-co-glycolic acid, polydioxanone, polycaprolactone, polycarbonates, polyorthoesters, polyamino acids, polyanhydrides, polyhydroxybutyrate, polyhydroxyvalyrate, poly (propylene glyco-co-fumaric acid), tyrosine-based polycarbonates; pharmaceutical tablet binders; cellulose, ethyl cellulose, micro-crystalline cellulose and blends thereof; and combinations of the foregoing.

9. The method of claim 1, wherein said forming step comprises depositing said composition into a mold comprising said predetermined shape, and storing said composition in said mold for a sufficient amount of time to allow for said composition to retain said predetermined shape.

10. The method of claim 9, wherein slight or no pressure is applied to said composition during said forming step.

11. The method of claim 10, wherein slight pressure comprises about 975 or less psi.

12. The method of claim 11, wherein slight pressure comprises between about 0 and about 500 psi.

13. The method of claim 9 wherein applying pressure to said composition is not required for said composition to retain said predetermined shape.

14. The method of claim 9, wherein the porosity of said osteogenic, implant is increased by applying less than about 975 psi to said composition during said forming step.

15. The method of claim 1, wherein said forming step comprises casting said composition into a pre-finished shape, and machining said pre-finished shape into a finished shape.

16. The method of claim 1, wherein said predetermined shape is selected from the group consisting of a sheet, plate, disk, cone, suture anchor, pin, wedge, cylinder, screw, tube or lumen, or dowel.

17. The method of claim 16 wherein said osteogenic, cross-linked implant has one or more threads, grooves, ridges, slots, holes, apertures, or furrows, or combinations thereof, machined on the surface thereof.

18. An osteogenic, cross-linked, composite implant produced according to the method of claim 1.

19. An osteogenic, cross-linked, composite implant comprised of fully mineralized, or partially or fully demineralized bone particles, or a combination thereof that are molded and cast into a predetermined shape through application of less than about 975 psi.
